(19) United States
(12) Patent Application Publication
(10) Pub. No.: US 2013/0071918 A1
(43) Pub. Date: Mar. 21, 2013

(54) CELL CULTURE CONTAINER AND METHOD FOR FABRICATING THE SAME

(71) Applicants: Dong Sung Kim, Pohang-si (KR); Kwang Sook Park, Hongseong-gun (KR); Dong-Woo Cho, Seoul (KR); Moonwoo Rha, Chuncheon-si (KR); Kyoung Je Cha, Daegu (KR); Bong-Kee Lee, Pohang-si (KR); Soo Hong Lee, Seoul (KR); Sun Woong Kang, Seoul (KR)

(72) Inventors: Dong Sung Kim, Pohang-si (KR); Kwang Sook Park, Hongseong-gun (KR); Dong-Woo Cho, Seoul (KR); Moonwoo Rha, Chuncheon-si (KR); Kyoung Je Cha, Daegu (KR); Bong-Kee Lee, Pohang-si (KR); Soo Hong Lee, Seoul (KR); Sun Woong Kang, Seoul (KR)

(73) Assignee: Postech Academy-Industry Foundation, Pohang-city (KR)

(21) Appl. No.: 13/634,299

(22) PCT Filed: Mar. 31, 2011
(86) PCT No.: PCT/KR11/02228

§ 371 Date: Sep. 12, 2012

(30) Foreign Application Priority Data
Jun. 10, 2010 (KR) 10-2010-0054988

(51) Int. Cl. C12M 1/22; B29C 45/00; B32B 37/10

(52) U.S. Cl. CPC C12M 23/10; B32B 37/10; B29C 45/0055
USPC 435/305.1; 156/247; 156/220; 264/219

(57) ABSTRACT

The present invention relates to a cell culture container, which enhances the efficiency of proliferation and differentiation of various cells including stem cells. The cell culture container according to an exemplary embodiment of the present invention includes a cell culture surface for adhering adult stem cells thereon to proliferate and differentiate the adult stem cells, and the cell culture surface includes a protrusion having a lotus leaf surface reproduction structure, which is disposed on the cell culture surface.

### TECHNICAL FIELD

The present invention relates to a cell culture container and a method for fabricating the same. More particularly, the present invention relates to a cell culture containing including a hydrophobic culture surface and a method for fabricating the same.

### BACKGROUND ART

As cell therapies using culture cells for the treatment of diseases currently expand, interests in cell culture are also increasing. Various devices are associated with the culture system, and one of the important factors for culturing cells is a cell culture container. In general, in order to obtain a large number of cells, cells are cultured in a cell culture container such as an artificially fabricated culture dish, a culture flask, a roller bottle and the like according to characteristics of cultured cells.

Cells artificially cultured are usually attached to the bottom of a cell culture container and persist while experiencing the process of growth, proliferation and differentiation. However, some cells proliferate while forming a plurality of layers and being overlapped on other cells, and some other cells also grow, proliferate and differentiate while being floated in a cell culture medium.

In this way, artificially fabricated cell culture containers have surface characteristics different from extracellular matrix in which cells originally reside, and thus cell proliferation and differentiation efficiency may be deteriorated. Actually, various cells are subjected to artificial proliferation and then used in clinical therapies. However, there are problems in that the differentiation induction of various cells including stem cells and the like for the treatment of patients and the like is not easily achieved.

The above information disclosed in this Background section is only for enhancement of understanding of the background of the invention and therefore it may contain information that does not form the prior art that is already known in this country to a person of ordinary skill in the art.

### DISCLOSURE

### Technical Problem

The present invention has been made in an effort to provide a cell culture container having advantages of enhancing the proliferation and differentiation efficiency of various cells including stem cells.

Further, the present invention has been made in an effort to provide a provides a method for fabricating a cell culture container, which reduces costs required for inducing the proliferation and differentiation of cells.

### Technical Solution

An exemplary embodiment of the present invention provides a cell culture container including a cell culture surface for adhering adult stem cells thereon to proliferate and differentiate the adult stem cells, and the cell culture surface includes a protrusion having a lotus leaf surface reproduction structure, which is disposed on the cell culture surface.

The cell culture surface may be formed integrally with the cell culture container.

The cell culture surface may be formed of a biocompatible polymer.

The biocompatible polymer may be any one of polysterene (PS), polymethyl methacrylate (PMMA), cyclic olefin copolymer (COC), polycarbonate (PC), polytetrafluoroethylene (PTFE), polydimethylsiloxane (PDMS), polyvinylchloride (PVC), polyurethanes (PU) and polyethylene terephthalate (PET).

Another exemplary embodiment of the present invention provides a method for fabricating a cell culture container, including: sequentially stacking a first conductive layer and an adhesive layer on a substrate, stacking a lotus leaf on the adhesive layer, sequentially stacking a second conductive layer and a metal plating layer on the lotus leaf, and sequentially separating the substrate and the lotus leaf to form a mold with a protrusion having a lotus leaf surface reproduction structure formed on one surface of the metal plating layer.

The method for fabricating a cell culture container according to the exemplary embodiment may further include using the mold to form a cell culture surface by a hot embossing process and attaching the cell culture surface to one surface of the cell culture container.

The method may further include disposing a metal pattern including a cavity having a shape of the cell culture container according to the embodiment and in which the mold is attached to one surface of the cavity, injecting a thermoplastic resin into the cavity and withdrawing a cell culture container formed while the thermoplastic resin is cured.

The cell culture container according to another exemplary embodiment of the present invention includes a cell culture surface for adhering adult stem cells to proliferate and differentiate the adult stem cells, and the cell culture surface includes a first protrusion formed with a diameter of from 10 μm to 15 μm and a height of from 10 μm to 20 μm and disposed at an interval of from 10 μm to 20 μm and a second protrusion formed on the micro protrusion and formed with a size of from 0.1 μm to 1 μm.

The first protrusion may have a conical or cylindrical shape.

### Effects of the Invention

According to exemplary embodiments of the present invention, effects on the proliferation and differentiation of cells may be to induce the cells to differentiate into certain cells or enhance the efficiency thereof.

Further, mass production of a cell culture container including a protrusion having a lotus leaf surface structure may be achieved, thereby reducing costs and time for cell culture.

### DESCRIPTION OF DRAWINGS

FIG. 1 is a schematic view illustrating a cell culture container according to an exemplary embodiment of the present invention.

FIGS. 2A and 2B are photos obtained by magnifying the cell culture surface of a cell culture container according to an exemplary embodiment of the present invention.

FIGS. 3A to 3E are views sequentially illustrating a fabrication process of a surface having a lotus leaf structure according to an exemplary embodiment of the present invention.

FIG. 4 is a view illustrating a fabrication process of a surface having a lotus leaf structure according to another exemplary embodiment of the present invention.

FIG. 5 is a photo illustrating the comparison of adhesion forces of adipose-derived stem cells.

FIG. 6 is a photo of the forms of adipose-derived stem cells observed by a fluorescent microscope.

FIG. 7 is a graph comparing the sizes of adipose-derived stem cells attached.

FIG. 8 is a photo illustrating staining and gene expression associated with the differentiation of adipose-derived stem cells into adipose cells.

### MODE FOR INVENTION

Hereinafter, an exemplary embodiment of the present invention will be described with reference to the accompanying drawings for those skilled in the art to easily implement the present invention. The size and thickness of each component shown in the drawings are arbitrarily shown for ease of the description, but the present invention is not always limited thereto.

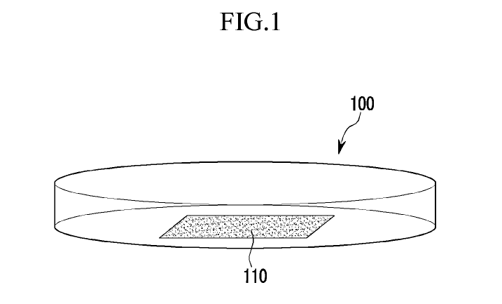

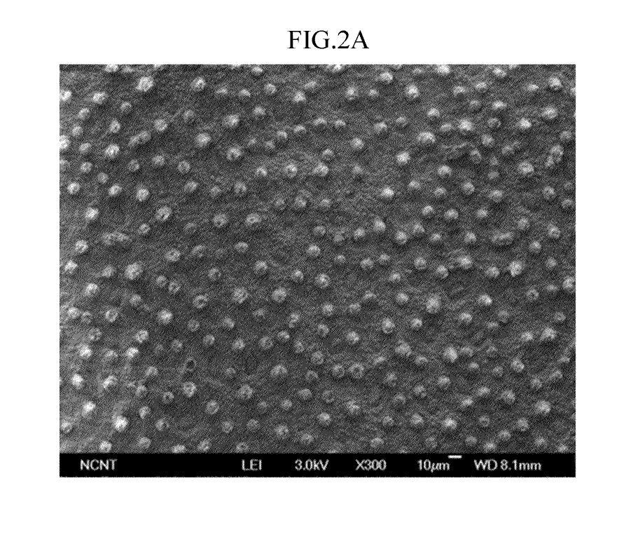

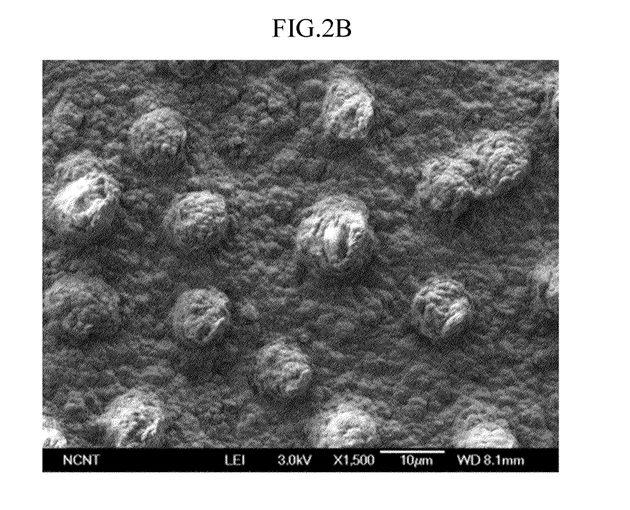

FIG. 1 is a schematic view illustrating a cell culture container according to an exemplary embodiment of the present invention, and FIGS. 2A and 2B are photos obtained by magnifying the cell culture surface of a cell culture container according to an exemplary embodiment of the present invention.

Referring to FIG. 1, a cell culture container 100 according to the present exemplary embodiment includes a cell culture surface 110. The cell culture surface 110 is for proliferating and differentiating cells artificially and allows cells to be cultured to be adhered on the cell culture surface 110 to induce the cells to differentiate into a desired direction. Examples of adult stem cells include bone marrow-derived stem cells, placenta-derived stem cells, adipose-derived stem cells and the like, and among them, adipose-derived stem cells may be obtained in a relatively large amount and have multipotency, and thus the cells may be variously utilized in regenerative medicine, tissue engineering and the like. The cell culture container 100 according to the present exemplary embodiment is for improving the attachment, proliferation and differentiation efficiencies of these adult stem cells, in particular, adipose-derived stem cells.

Referring to FIGS. 2A and 2B, in the cell culture surface 110 of the cell culture container 100 according to the present exemplary embodiment, one surface on which cells are adhered is formed of a lotus leaf surface reproduction structure. That is, one surface of the cell culture surface 110 is formed while protrusions having the same form as the surface of the lotus leaf are irregularly disposed, and each protrusion has a micro size and is minutely formed.

Specifically, the protrusion of a lotus leaf surface reproduction structure on the cell culture surface 110 may be a micro protrusion formed with a diameter of from about 10 μm to about 15 μm and a height of from about 10 μm to about 20 μm. Moreover, these micro protrusions may be disposed at an interval of from about 10 μm to about 20 μm on the cell culture surface 110. In this case, the micro protrusion may be formed in a conical or cylindrical shape. Meanwhile, a nano protrusion having a size of from about 0.1 nm to about 1 nm may be further formed on the micro protrusion.

In the present exemplary embodiment, the cell culture surface 110 is formed of polystyrene (PS), but the present invention is not limited thereto. The material for the cell culture surface 110 may be variously changed by a person of an ordinary skill in the art. Specifically, the cell culture surface 110 is sufficient if the cell culture surface 110 is formed of a biocompatible polymer, and may be formed of polymethyl methacrylate (PMMA), cyclic olefin copolymer (COC), polycarbonate (PC), polytetrafluoroethylene (PTFE), polydimethylsiloxane (PDMS), polyvinylchloride (PVC), polyurethanes (PU) or polyethylene terephthalate (PET) besides polystyrene.

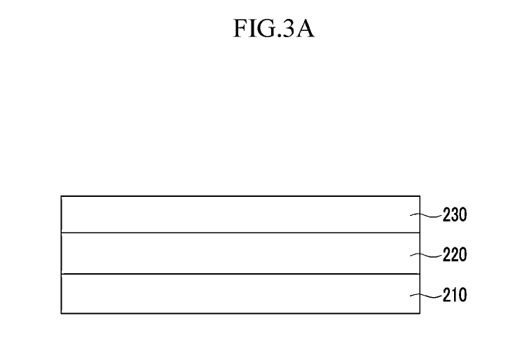

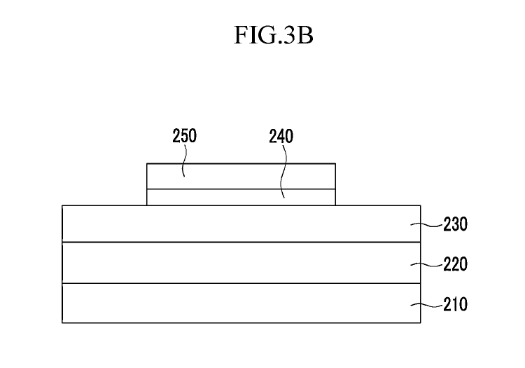

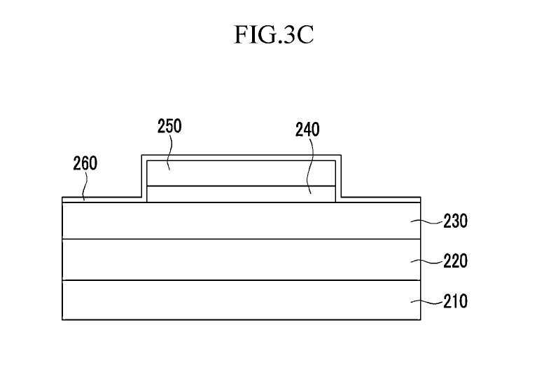

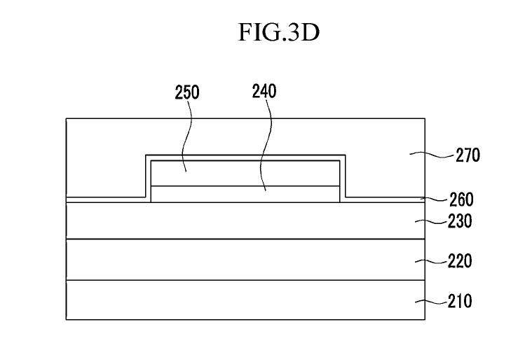

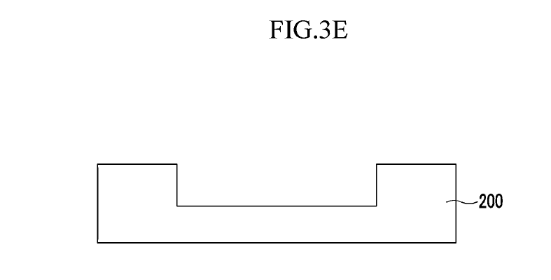

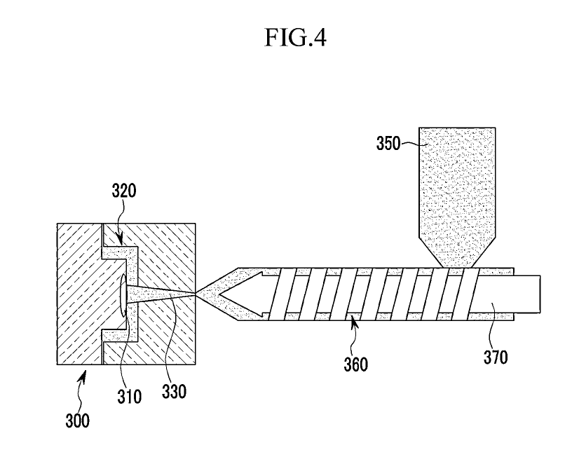

FIGS. 3A to 3E and FIG. 4 are views sequentially illustrating a fabrication process of a cell culture container according to an exemplary embodiment of the present invention, and referring to the views, a method for fabricating a cell culture container according to an exemplary embodiment of the present invention will be described.

Referring to FIG. 3A, an adhesion enhancing layer 220 is stacked on a substrate 210 and a first conductive layer 230 is stacked on the adhesion enforcing layer 220. The adhesion enforcing layer 220 is for improving adhesion force between the substrate 210 and the first conductive layer, and is formed by including an adhesion enhancing material such as chromium, titanium or the like. Meanwhile, as the substrate 210, a silicon substrate such as a silicon wafer is used, and the first conductive layer 230 is formed by including a conductive material such as gold, copper, nickel or the like.

Referring to FIGS. 3B and 3C, an adhesive layer 240 is formed on the first conductive layer 230, then a lotus leaf 250 is attached thereto, and a second conductive layer 260 is stacked so as to cover the lotus leaf 250 and the first conductive layer 230. In this case, an epoxy-based adhesive may be used on the adhesive layer 240, and the second conductive layer 260 is formed by a process of coating gold or carbon ions.

Referring to FIG. 3D and FIG. 3E, a plating layer 270 is formed on the second conductive layer 260 through a plating process. In the present exemplary embodiment, a metal used in the plating process includes any one of nickel, copper, silver, gold and an alloy of zinc tin-lead. A mold 200 for forming a cell culture surface having a lotus leaf surface structure is formed by forming the plating layer 270, removing the substrate 210 and then removing the adhesion enforcing layer 220 which is in contact with the plating layer 270, the first conductive layer 230, the second conductive layer 260 and the lotus leaf 250. A reverse pattern of the lotus leaf surface is reproduced and formed on one surface of the mold 200, and through this, a cell culture surface having a protrusion of a lotus leaf surface structure may be formed. Meanwhile, in the present exemplary embodiment, materials for the substrate 210, the adhesion enforcing layer 220, the first conductive layer 230, the adhesive layer 240, the second conductive layer 260 and the plating layer 270 have been described with limitation thereto. However, the present invention is not limited thereto and these materials may be variously changed by a person of an ordinary skill in the art.

A cell culture container is fabricated by forming the mold 200 through the process, and then forming a cell culture surface through a hot embossing process to attach the cell culture surface to one surface of the cell culture container. The hot embossing process is a process for using a thermoplastic resin to fabricate a structure having a small size from micrometer to nanometer, and in the present exemplary embodiment, polystyrene (PS) is heated and then the mold 200 is pressurized on the polystyrene to form a cell culture surface. As described above, a reverse pattern of a lotus leaf surface is formed on one surface of the mold 200, and thus one surface of a cell culture surface formed by a hot embossing process using the mold 200 has the same surface as the lotus leaf surface. Meanwhile, even in the present exemplary embodiment, various biocompatible polymers such as polymethyl methacrylate and the like besides polystyrene may be used as a material for forming a cell culture surface.

A method for fabricating a cell culture container has been described in a manner that the mold 200 is used to form a cell culture surface separately, and then the cell culture is attached to one surface of the cell culture container, the cell culture surface may be formed integrally with the cell culture container, as in FIG. 4.

Referring to FIG. 4, a metal pattern 300 including a cavity 320 having a cell culture container shape is disposed, and a mold 310 including a lotus leaf surface and a reverse pattern, which have been fabricated in a manner as in FIGS. 3A to 3E, is disposed at a position where the cell culture surface is formed. In disposing the mold 310, it is considered that the lotus leaf surface structure needs to be formed on one surface of the cell culture container. Subsequently, a thermoplastic resin, which is a molding material for a cell culture container, that is, polystyrene is supplied into a cylinder 360 from a hopper 350, and the thermoplastic resin is heated in the cylinder 360 through a heater (not shown) and becomes a fluid state. A fluid state thermoplastic resin passes through a screw DeletedTexts a nozzle and is injected into the cavity 320 of the metal pattern 300 through an injection port 330, and after the injection is completed, polystyrene is cooled, thereby completing the fabrication of a cell culture container.

When the cell culture container is fabricated in this manner, a cell culture surface formed of a lotus leaf surface structure may be integrally formed, and thus the process is simplified and time and costs may be reduced.

Effects obtained when stem cells are cultured with a cell culture container including a cell culture surface having a lotus leaf surface structure according to the present exemplary embodiment will be described in detail below through Experimental Examples.

### Experimental Example 1

First, a cell culture surface having a lotus leaf surface structure was fabricated in the same manner as in the method described through FIGS. 3A to 3E.

First, chromium and gold were deposited as an adhesion enhancing layer and a first conductive layer on a silicon wafer, and a lotus leaf was attached thereon. Next, gold was deposited as a second conductive layer, nickel having a thickness of 1 mm or more was formed through a plating process, the nickel layer was separated from the wafer, and then the lotus leaf was removed to fabricate a mold. A nickel mold of a reverse pattern of the lotus leaf surface fabricated and polystyrene were used to form a cell culture surface on which the lotus leaf surface shape was copied through a hot embossing process. Specifically, trichlorosilane was deposited on the surface of the nickel mold by using a vacuum deposition method, the temperature was increased to about 110° C. while polystyrene was placed on the nickel mold, a pressure of about 3.2 MPa was applied for 10 min, and finally the nickel mold was cooled to 45° C. Through this process, as shown in FIGS. 2A and 2B, a cell culture surface having a lotus leaf surface structure, on which a protrusion shape having a size of from 10 μm to 15 μm and a shape having a sub-micro size are molded, was fabricated, and the cell culture surface was attached to one surface of a cell culture container to fabricate the cell culture container.

In order to increase the attachment efficiency of adipose-derived stem cells to the cell culture container thus fabricated, oxygen plasma treatment was performed, and then the adipose-derived stem cells were attached thereto and cultured. Meanwhile, as Comparative Example for evaluating effects on the attachment efficiency, adipose-derived stem cells were attached to a cell culture container including a flat cell culture surface, and then cultured.

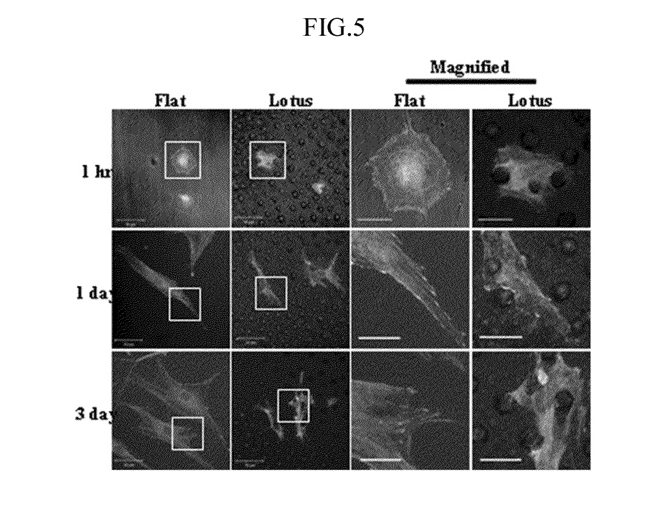

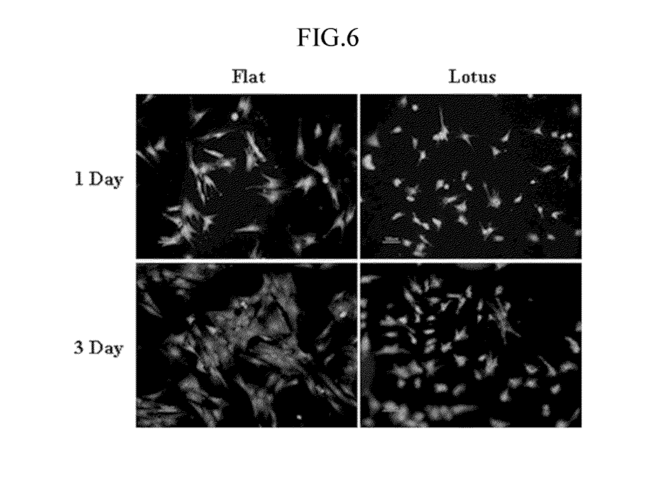

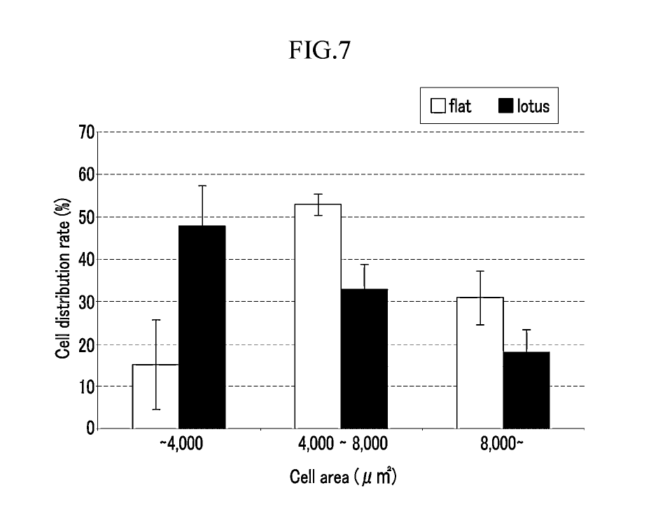

FIGS. 5 and 6 are photos illustrating focal adhesion and shapes of adipose-derived stem cells after the adipose-derived stem cells had been cultured for 3 days in the present exemplary embodiment and Comparative Example, respectively, and FIG. 7 is a graph showing the comparison of sizes of adipose-derived stem cells. Referring to the drawings, it can be confirmed that the cytoskeleton had been formed between protrusions having a size of about 10 μm, and vinculin which is a protein associated with focal adhesion was positioned at the edge of protrusions having a size of about 10 μm or protrusions of a sub-micro size. Furthermore, it can be confirmed that the ratio of cells showing that adipose-derived stem cells had been widely dispersed was high in Comparative Example, while the ratio of cells showing that cells had been relatively narrowly distributed was high in the present exemplary embodiment.

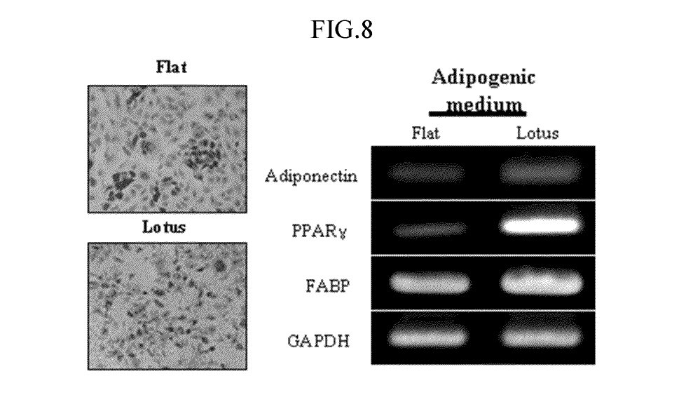

FIG. 8 is a photo showing the staining and gene expression of adipose-derived stem cells when the cells had been cultured for from 2 weeks to 3 weeks in the present exemplary embodiment and Comparative Example, respectively. Specifically, the photo is a photo examining effects of using adipogenic, chondrogenic and osteogenic induction media on differentiation potency in the present exemplary embodiment and Comparative Example. Referring to this, it can be confirmed that the expression of genes in the drawing of the present exemplary embodiment had significantly increased compared to Comparative Example. In particular, it can be confirmed that the expression of genes associated with the differentiation of adipose cells has significantly increased.

As described above, when adipose-derived stem cells are cultured in a cell culture container including a cell culture surface having a lotus leaf surface structure according to the present exemplary embodiment, effects that cell adhesion, proliferation and differentiation have been stably induced may be obtained. Further, when adipose-derived stem cells are cultured through this structure, the efficiency thereof in cell differentiation, particularly, differentiation of adipose cells will increase and a large number of cells may be obtained.

The present invention has been described through preferred embodiments, but, the present invention is not limited thereto. The scope of the present invention is determined by the description of the claims, and it is to be easily understood by those skilled in the art, to which the present invention pertains, that various modifications and changes can be made without departing from the concept and scope of the claims.

1. A cell culture container, comprising: a cell culture surface for adhering adult stem cells thereon to proliferate and differentiate the adult stem cells, wherein the cell culture surface includes a protrusion having a lotus leaf surface reproduction structure, which is disposed on the cell culture surface.

2. The cell culture container of claim 1, wherein:
the cell culture surface is formed integrally with the cell culture container.

3. The cell culture container of claim 1, wherein:
the cell culture surface is formed of a biocompatible polymer.

4. The cell culture container of claim 3, wherein:
the biocompatible polymer is any one of polysterene (PS), polymethyl methacrylate (PMMA), cyclic olefin copolymer (COC), polycarbonate (PC), polytetrafluoroethylene (PTFE), polydimethylsiloxane (PDMS), polyvinylchloride (PVC), polyurethanes (PU) and polyethylene terephthalate (PET).

5. A method for fabricating a cell culture container, including: sequentially stacking a first conductive layer and an adhesive layer on a substrate and stacking a lotus leaf on the adhesive layer;
sequentially stacking a second conductive layer and a metal plating layer on the lotus leaf; and
sequentially separating the substrate and the lotus leaf to form a mold with a protrusion having a lotus leaf surface reproduction structure formed on one surface of the metal plating layer.

6. The method of claim 5, further comprising:
using the mold to form a cell culture surface by a hot embossing process; and
attaching the cell culture surface to one surface of the cell culture container.

7. The method of claim 5, further comprising:
disposing a metal pattern including a cavity having a shape of the cell culture container and in which the mold is attached to one surface of the cavity;
injecting a thermoplastic resin into the cavity; and
withdrawing a cell culture container formed while the thermoplastic resin is cured.

8. A cell culture container, comprising: a cell culture surface for adhering adult stem cells thereon to proliferate and differentiate the adult stem cells,
wherein the cell culture surface comprises,
a first protrusion formed with a diameter of from 10 μm to 15 μm and a height of from 10 μm to 20 μm and disposed at an interval of from 10 μm to 20 μm; and
a second protrusion formed on the micro protrusion and formed with a size of from 0.1 μm to 1 μm.

9. The cell culture container of claim 8, wherein:
the first protrusion has a conical or cylindrical shape.
